(19) United States
(12) Patent Application Publication
(10) Pub. No.: US 2015/0238958 A1
(43) Pub. Date: Aug. 27, 2015

(54) MICROFLUIDIC DEVICE FOR BIOLOGICAL SAMPLE PREPARATION

(71) Applicants: Frederic ZENHAUSERN, Fountain Hills, AZ (US); Matthew ESTES, Phoenix, AZ (US); Jianing YANG, Tempe, AZ (US); ARIZONA BOARD OF REGENTS FOR THE UNIVERSITY OF ARIZONA, Tucson, AZ (US)

(72) Inventors: Frederic Zenhausern, Chandler, AZ (US); Matthew Estes, Chandler, AZ (US); Jianing Yang, Chandler, AZ (US)

(21) Appl. No.: 14/428,218

(22) PCT Filed: Sep. 13, 2013
(86) PCT No.: PCT/US2013/059752

§ 371 Date: Mar. 13, 2015

Related U.S. Application Data
(60) Provisional application No. 61/743,935, filed on Sep. 14, 2012.

(51) Int. Cl. B01L 3/00; C12Q 1/68; C12N 15/10

(52) U.S. Cl. CPC B01L 3/502707; C12N 15/1003; C12Q 1/6806; B01L 2300/0867; B01L 2400/0415

(57) ABSTRACT

The present invention provides a microfluidic device for preparing or extracting a biological sample from a solid material and methods for using the same. The microfluidic device of the invention automates the biological sample extraction from a solid material, thereby significantly reducing the possibility of contamination, human error and a potential variability of the sample that is obtained by different technicians.

### CROSS-REFERENCE TO RELATED APPLICATIONS

This application claims the priority benefit of U.S. Provisional Application No. 61/743,935, filed Sep. 14, 2012, which is incorporated herein by reference in its entirety.

### FIELD OF THE INVENTION

The present invention relates to a microfluidic device for preparing or extracting a biological sample from a material for analysis and methods for using the same. While a biological sample from a wide variety of materials can be obtained for analysis using the device and the method of the invention, the device of the invention is particularly useful in obtaining a biological sample from a solid material.

### BACKGROUND OF THE INVENTION

Currently, commercially available integrated forensic analysis instrumentations for sample preparation for analysis (e.g., by capillary electrophoresis or “CE” processing) are incapable of solid-phase DNA extraction. Conventional analysis instruments require preprocessed samples before they can be analyzed. At present, samples are preprocessed for analysis by highly skilled technicians. Even though most skilled technicians are quite capable of properly processing a sample for analysis, there still is a chance for human error and contamination of the sample, as well as a potential inconsistency from one technician to another. Such variations or errors can result in unreliable or even false sample data, which can potentially have a huge impact on the person whose sample is being analyzed. Moreover, sample processing by a technician is increases the time and cost for sample analysis.

In order to reduce or eliminate human errors and potential contamination as well as a potential variability of processed sample from one technician to another, it would be useful to automate the sample preparation process.

### SUMMARY OF THE INVENTION

Some aspects of the invention provide a microfluidic solid material processing device and methods for using the same. In one particular embodiment, the device of the invention allows the integration of DNA extraction from a solid biological sample with further downstream microfluidic processing. However, it should be appreciated that the device of the invention can be used for processing a wide variety of samples for analysis.

In one particular aspect of the invention, a microfluidic sample preparation device (100) is provided. In one embodiment of the invention, the microfluidic device 100 comprises:
a material processing chamber (200) adapted for extracting a biological sample using a solvent, said sample processing chamber (200) comprising:
   a material inlet port (204);
   a solution inlet port (208); and
   a processed sample solution outlet port (212);
a solution chamber (216) adapted for holding a fluid comprising:
   a solution delivery microfluidic channel (220) operatively connected to said solution inlet port (208), wherein said solution delivery microfluidic channel (220) is adapted for delivering a sample processing solution from said solution chamber (216) to said sample processing chamber (200);
a sample solution metering element (224) adapted for metering a desired volume of the sample solution to a sample analyzer, wherein said sample solution metering element (224) comprises:
   a sample solution delivery microfluidic channel (228) operatively connected to said sample solution outlet port (212), wherein said sample solution delivery microfluidic channel (228) is adapted to provide a desired volume of the sample solution to a sample analyzer;
an excess sample solution storage chamber (232) operatively connected to said sample solution metering element (224), wherein said excess sample solution storage chamber (232) is adapted to store any excess portion of the sample solution; and a pump element (244).

It should be appreciated, the pump element 244 can be a separate device or a unit or it can be integrated within the microfluidic channels as discussed in detail below.

In some embodiments, said solution chamber (216) further comprises a solution chamber valve system for opening (236A) and closing (236B) said solution delivery microfluidic channel (220).

Yet in other embodiments, said sample solution metering element (224) further comprises a sample solution metering valve system (240A and 240B) operatively connected to said sample processing chamber (200).

Still in other embodiments, the microfluidic device 100 further comprises an electrochemical pump (244) that is operatively connected to said solution chamber (216) and is adapted to transfer a fluid from said solution chamber (216) to said sample processing chamber (200).

In other embodiments, the microfluidic device 100 further comprises an electrochemical pump (244) that is operatively connected to said sample processing chamber (200) and is adapted to transfer the sample solution from said sample processing chamber (200) to said sample solution metering element (224). It should be appreciated that the microfluidic device 100 can have a single pump 244 that can be used to transfer the solution to the material processing chamber and to transfer the sample solution to the metering element 224. Alternatively, the microfluidic device can have a plurality of pumps, each of which can be used for a various purposes.

Yet still in other embodiments, said sample processing chamber (200) further comprises a vent (248). The vent 248 can be operatively connected via a microfluidic channel 264 or it can be present on the material processing chamber 200.

Still yet in other embodiments, the microfluidic device 100 is a cartridge. In this manner, each microfluidic device 100 can be used once and discarded, thereby eliminating a likelihood of material to material contamination.

In other embodiments, said sample processing chamber (200) is adapted to provide an active mixing of the sample and the sample processing solution. This can be achieved, for example, by activating the pump 244 to allow air or other suitable gas (e.g., an inert gas such as nitrogen, argon, or helium) to enter the material processing chamber 200 through the solution inlet port 208 while keeping the vent 248 open.

Another aspect of the invention provides a method for processing a solid material comprising a biological sample using a microfluidic device of the invention to obtain the biological sample therefrom. Typically, such a method comprises:
(i) placing a solid material comprising a biological sample in said sample processing chamber (200) via said sample inlet port (204);
(ii) activating said solution delivery microfluidic channel (220) thereby transferring a sample processing solution from said solution chamber (216) to said sample processing chamber (200);
(iii) processing the sample in said sample processing chamber (200) under conditions sufficient to produce a desired biological sample solution from the solid material; and
(iv) activating said sample solution delivery microfluidic channel (228) thereby transferring the sample solution to said sample solution metering element (224).

In some embodiments, said steps (ii)-(iv) are automated.

Still in other embodiments, the method can further comprise the step of transferring an excess sample solution to said excess sample solution storage chamber (232).

Yet in other embodiments, said microfluidic device further comprises a pump (244) that is operatively connected to said solution chamber (216), and wherein said step (ii) of activating said solution delivery microfluidic channel (220) comprises activating said pump (244) thereby transferring the fluid from said solution chamber (216) to said material processing chamber (200) through said solution delivery microfluidic channel (220).

In other embodiments, said microfluidic device further comprises a pump (244) that is operatively connected to said material processing chamber (200), and wherein said step (iv) of activating said sample solution delivery microfluidic channel (228) comprises activating said pump (244) thereby transferring the sample solution from said material processing chamber (200) to said sample solution metering element (224) through said sample solution delivery microfluidic channel (228).

Still yet in other embodiments, said microfluidic device is a cartridge, thereby allowing a single use application, which eliminates any possibility of material-to-material contamination.

Yet in other embodiments, the method can further comprise the steps of transferring the sample solution in said sample solution metering element (224) to an analytical device to analyze said sample solution.

### BRIEF DESCRIPTION OF THE DRAWINGS

FIG. 1A is a schematic drawing of one embodiment of the stand alone microfluidic sample preparation device of the invention.

FIG. 1B is a schematic drawing of one embodiment of the integrated microfluidic sample preparation device of the invention.

FIG. 2 is a detailed schematic of the material processing chamber dimensions in mm of one particular embodiment of the present invention.

FIG. 3 shows use of the microfluidic device of the invention in Mini-MiDAS instrument.

FIG. 4 is a schematic drawing of one particular embodiment of a microfluidic device of the invention suitable for swab sample processing that includes swab lysis module, DNA extraction module, multiplex PCR module and transfer module delivering PCR product to a capillary electrophoresis (CE) microchip for CE detection.

### DETAILED DESCRIPTION OF THE INVENTION

Since its inception in the field of forensic science in 1985, human identification using an individual-specific DNA “fingerprint” generated from multiple highly variable genetic loci simultaneously, e.g., polymorphic short tandem repeat (STR), has been a powerful tool widely used for forensic DNA analysis, DNA data basing, paternity testing, missing person identification, clinical specimen identification and cell line authentication. The higher power of discrimination and ability to obtain STR profiles from all types of forensic samples dramatically increased the amount of evidence samples flowing into forensic laboratories where a large backlog of samples have been waiting for processing.

Traditionally, Short Tandem Repeat (STR) analysis for human identification is a lengthy process with a cycle time that can last up to several days or weeks, from sample collection, extraction of DNA from a biological sample, quantification of extracted DNA, multiplex amplification of STR loci, the separation of amplified products, the analysis of data, to the result reporting, while requiring at least 8-11 hours in laboratory processing by skilled personnel to complete the process under routine conditions. Furthermore, emerging developments in biometrics also comprise complementary bio-signatures, such as the use of Single Nucleotide Polymorphisms (SNP) that can provide additional phenotypic information for refining human identification. As the result of advancement in technology and chemistries, lab-on-a-chip types of portable, mobile, integrated, automated, and multiplexing systems for rapid forensic DNA STR genotyping have been rapidly developed. All of the integrated systems currently existing are cartridge based, functionally multi-modular platforms, with microfluidic integration of multiple analysis steps combining DNA extraction, PCR amplification, electrophoretic separation and detection on a single micro device.

The concept of micro-total analysis system (μTAS) was first described more than a decade ago. Since then, various integrated microfluidic systems with “sample-in and answer-out” capability were designed, tested, and demonstrated in pathogen detection, clinical application and forensic samples. The integrated microfluidic systems reported for pathogen or infectious disease detection were capable of performing target cell pre-concentration, cell lysis, reagents mixing, PCR, separation and detection with either microarray platform, laser-excited fluorescence signal, separating PCR products with gel electrophoresis or on microchip electrophoresis. However, the “sample-in” reported in these pathogen detection microfluidic devices were all limited to one type of pathogen and involved in only single-plex PCR reaction. The sample solutions were introduced by manually pipette-in or delivered in through a syringe pump. As for an integrated micro device for forensic DNA analysis, a prospective design with a “sample-in” capability was first described several years ago.

At present, most of the integrated rapid DNA analysis systems in forensic science reported require initial bench-top processing of collected evidence samples, i.e. off-chip swab lysis, to get them into a liquid lysate form, suitable for microfluidic workflow processing. Therefore, the integrated systems start with sample lysate. This off-chip lysis step poses a significant restriction in achieving a fully-integrated microfluidic device which is sample-in to answer-out. More and more effort has been focused on achieving a total integrated analytical system with a direct swab-in method, not only in the forensic laboratories but also for commercial streamlined robotic sample processing equipment.

Forensic casework samples are varied, ranging from bloodstain swabs, cigarette butts, chewing gum, sexual assault samples, buccal swabs or saliva, urine, fingernail swabs, to hair roots, tissues or bones, teeth, and swabs of shoes or other solid surfaces. Although different tools have been used for different sample types, the collection techniques have become more streamlined. Among all the forensic sample collection techniques, such as swabbing, cuttings (e.g., a cloth material), scrapings, tape lifts, or collectors, swabbing has been generally considered as more effective for collection and recovery of DNA as compared to taping and other methods. Swabs are typically used for sample collection systems, especially in the forensic community (e.g., Bode SecurSwab). Maximal collection and subsequent adequate recovery of the biological evidence samples from the crime scene is one of the first and crucial steps to leverage the benefits of forensic DNA technology.

Albeit these complex variables, the most commonly found collector still being used is the cotton swab. Cotton swabs are inexpensive, widely available, robust and well-established in the collection of biological evidence, lifting as much as 95% of particular samples such as buccal cells, vaginal samples, touch evidence, blood stains, saliva from human skin and even exploded pipe bomb devices. Therefore, swab lysis is the very first step to release the evidence material from the swab matrix and for the downstream nucleic acids extraction, Polymerase Chain Reaction (PCR) and STR profile analysis. While currently there are a few swab-in/profile-out integrated systems in the literature, demonstrating success with reference sample swabs, no detailed characterization of their swab lysis device, regarding to the on-chip lysis efficiency and capability was reported. Addition of a swab lysis cartridge module, which can process many types of forensic swab samples, to the integrated sample prep to detection platform is a critical step toward a total analysis system (μTAS).

As generally discussed above, forensic human identification based on a single solid phase biological sample generally requires four key steps: Extraction of DNA from the sample, purification of the DNA, amplification of the DNA, and analysis of the DNA. Automated rapid forensic DNA analysis requires integrating the multistep process onto a microfluidic platform, including the collection substrate lysis. While the extraction of DNA from the released lysate solution, multiplex PCR amplification of STR loci, separation of PCR products by capillary electrophoresis, and the result analysis for allelic peak calling have been automated, currently there is no reliable device for preparing a sample for analysis. In particular, most conventional integrated sample analysis systems start with the sample lysate and an off-chip lysis of collected substrates.

Some aspects of the invention provide a microfluidic device for sample preparation that is adapted for extracting a biological sample from a material. The device of the invention can be used for a wide variety of applications including, but not limited to, forensic human identification, pathogen or infectious disease detection, etc. Moreover, the device of the invention can be automated to prepare and/or extract a biological sample from any material, in particular from a solid material. Suitable solid material containing a biological sample that can be used in the device of the invention include, but are not limited to, swab (such as a cotton swab), a fabric, cigarette butts, chewing gum, sexual assault samples, hair roots, tissues, bones, teeth, and any other solid material that are typically used in forensic science to gather evidence or in medical diagnostics. For the sake of clarity and brevity, however, the device and the method of the invention will now be described in reference to preparing a biological sample for analysis from a swab.

One particular embodiment of the device of the invention will be described with regard to the accompanying drawings, which assist in illustrating various features of the invention. In this regard, the present invention generally relates to a microfluidic device for biological sample preparation. In one particular embodiment, the invention relates to a microfluidic device for obtaining (e.g., extracting) a biological material from a solid substance. The biological material thus obtained can be used in a wide variety of application including, but not limited to, forensic science (e.g., for identification purposes), pathogen and infectious disease detection, paternity test, molecular profiling of disease states such as cancer or cardiovascular diseases, but also genetic diseases, for example assessing spinal disc degenerative diseases and potential surgical treatment benefit of an individual, neurological or metabolic diseases, biodosimetry and testing for radiosensitivity and/or late toxicities of drug or radiation therapies, point of care testing of critical care medicine (e.g. pulmonary complications) etc. A typical biological material that is obtained using the device and the method of the invention include, but are not limited to biological fluids such as blood, saliva, cells or tissues, ascites and effusions (e.g., pleural effusions, cerebrospinal fluid), tears, feces, semen, lavage fluids (e.g. nasal, vaginal), and any other biological material that is suitable for analysis.

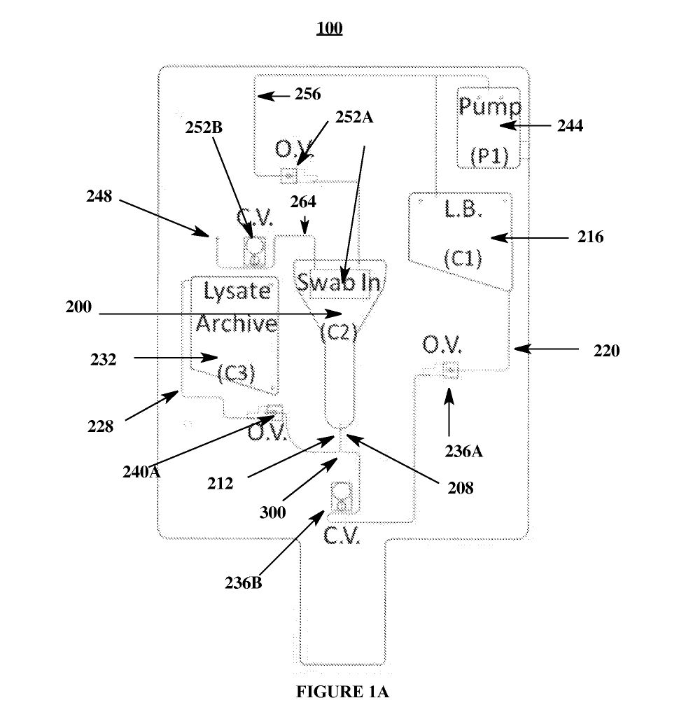

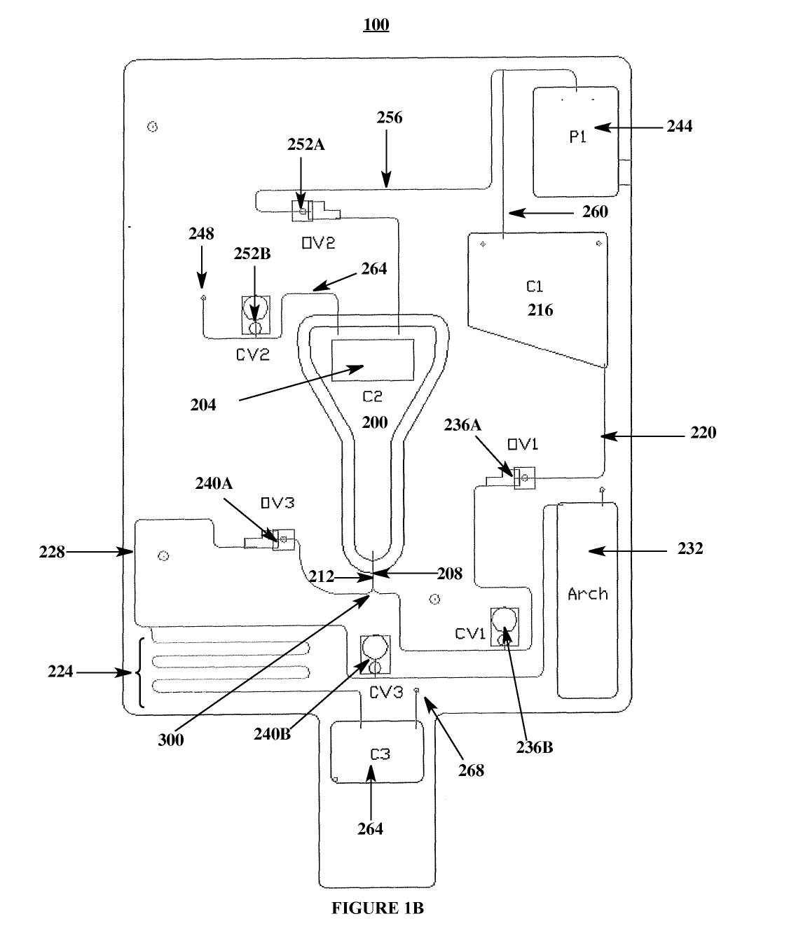

One particular embodiment of a microfluidic device for biological sample preparation is generally illustrated in FIGS. 1A and 1B, in which FIG. 1A is a standalone design and FIG. 1B is adapted to be integrated with other downstream processing of the sample solution. It should be appreciated that all accompanying drawings are provided solely for the purpose of illustrating the practice of the invention and does not constitute limitations on the scope thereof. As shown in FIGS. 1A and 1B, one particular embodiment of a microfluidic device 100 for biological sample preparation comprises a material processing chamber 200 adapted for obtaining or extracting a biological sample from a material. The material processing chamber 200 includes a material inlet port 204 that is adapted for placing the material into the microfluidic device 100; a solution inlet port 208; and a sample solution outlet port 212. In FIG. 1, the solution inlet port 208 and the sample solution outlet port 212 is the same except that there is a “T” junction 300 that controls the direction of the solution flow. However, it should be appreciated that the scope of the invention includes having a separate solution inlet port 208 and the sample solution outlet port 212.

In some embodiments, the material processing chamber 200 is adapted to allow active mixing of the material from which the biological sample is to be obtained and the elution or extraction solution that is used to separate the biological sample from the material. As used herein, the term “active mixing” refers to providing an agitation or a mixing action of the solution. It does not include a diffusion process where the material is placed in the solution without any movement of the solvent by an external force.

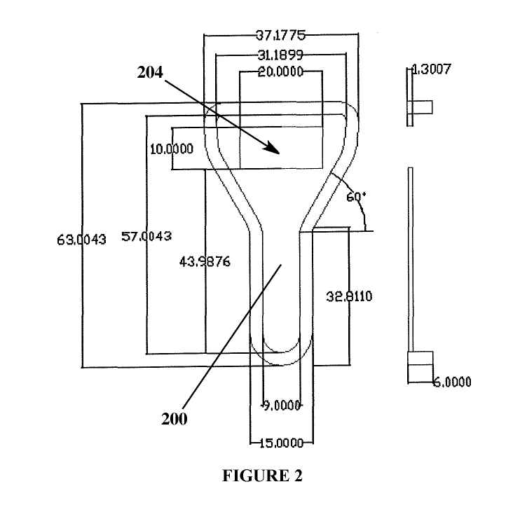

FIG. 2 shows a detailed schematic drawing of one particular embodiment of the material processing chamber 200, where the dimensions are presented in mm. In some instances, the three mm margin around the edge of the chamber allows for reliable sealing over a wide variety of temperatures. It should also be noted that the material processing chamber 200 is designed such that the solid material placed within the material processing chamber 200 does not flow into the sample solution outlet port 212. Such withholding of the solid material within the material processing chamber 200 can be achieved, for example, by tapering the material processing chamber 200 and/or by the size of the sample solution outlet port 212. Such a design serves to filter the solid material and allows transfer of only the sample solution through the sample solution outlet port 212.

Referring again to FIGS. 1A and 1B, the microfluidic device of the invention also includes a solution chamber 216 that is adapted for holding a fluid. The type and the amount of fluid in the solution chamber 216 can vary depending on the particular biological sample to be extracted and/or the material that contains the biological sample. Suitable solvents for a particular biological sample of interest are well known to one skilled in the art. In one particular embodiment, the solution chamber 216 is designed to hold about 1 mL of solution. The solution chamber 216 includes a solution delivery microfluidic channel 220 that is operatively connected to the solution inlet port 208. As used herein, the term “microfluidic channel” refers to a conduit that allows micro volume (e.g., nL to L) of fluid to flow through, typically from about 10 nL to 1 mL, often from about 100 nL to about 1 mL, and more often from about 1 μL to about 1 mL. The cross-sectional area of the microfluidic channel is typically about 0.1 μm², often about 10 μm², and more often about 100 μm². In some instances, the cross-sectional area of the microfluidic channel ranges from 1 nm² to about 1 mm², typically from 10 nm² to about 100 μm², and often from about 1 m² to about 100 μm². The cross-section of a microfluidic channel can be any shape including, but not limited to a circle, oval, rectangle, square, hexagon, octagon, etc. The solution delivery microfluidic channel 220 is adapted for delivering a biological sample extraction solution from the solution chamber 216 to the material processing chamber 200.

The microfluidic device 100 also includes a sample solution metering element 224 adapted for metering a desired volume of the sample solution to a sample analyzer (not shown). The sample solution metering element 224 includes a sample solution delivery microfluidic channel 224 operatively connected to the sample solution outlet port 212. The sample solution delivery microfluidic channel 228 is adapted to provide a desired volume of the sample solution to a sample analyzer (not shown).

The microfluidic device 100 also includes an excess sample solution storage chamber 232 operatively connected to the sample solution metering element 224. The excess sample solution storage chamber 232 is adapted to store any excess portion of the sample solution. The sample solution metering element 224 can be a microfluidic channel or a chamber of a predefined volume.

In some embodiments, the solution chamber 216 further comprises a solution chamber valve system (e.g., 236A and 236B) for opening and closing said solution delivery microfluidic channel 220.

Yet in other embodiments, the sample solution metering element 224 further comprises a sample solution metering valve system (e.g., 240A and 240B) operatively connected to the material processing chamber 200.

Still in other embodiments, the microfluidic device 100 further comprises a pump 244 or a device that is operatively connected to the solution chamber 216 and/or the material processing chamber 200. The pump 244 is adapted to transfer a fluid from the solution chamber 216 to the material processing chamber 200. The pump 244 also is adapted to transfer the sample solution from the material processing chamber 200 to the sample solution metering element 224. It should be appreciated that the pump 244 can be a single unit that operates both the material processing chamber 200 and the solution chamber 216. Alternatively, each of the material processing chamber 200 and the solution chamber 216 can have a separate pump unit.

In FIG. 1, the pump 244 is connected to the material processing chamber 200 by a microfluidic channel 256. In such instances, there can be a valve system 252A that can be used to control the flow of sample solution from the material processing chamber 200 to the sample solution metering element 224.

In some embodiments, the material processing chamber 200 further comprises a vent 248.

Yet in other embodiments, the vent 248 is connected to the material processing chamber 200 via a microfluidic channel 264. In such embodiments, there can be a valve system (e.g., 252B) that can be used to close the vent 248.

Still in other embodiments, the microfluidic device 100 is a cartridge. In this manner, each microfluidic device 100 can be used for processing a single material to obtain a biological sample for further processing.

It should be appreciated that the microfluidic channels can other elements of the microfluidic device 100 can be fabricated from an elastic polymer. Use of elastic polymers allows actuation of microfluidic channels or other elements, e.g., electrically or pneumatically. In some other embodiments, the elastic polymer is comprises of an electrically activated materials such that when it is activated, it closes (or alternatively opens) the microfluidic channel. By having a plurality of areas of a microfluidic channel that can be actuated, one can generate a peristaltic-like wave movement. Thus, the pump unit of microfluidic device 100 can comprise a plurality of areas of the microfluidic channel that can be actuated. In addition, it should be appreciated that any valve system (e.g., the solution chamber valve system and/or sample solution metering valve system) can be a single valve that can control opening and closing, e.g., by a pneumatic or electrical means. For example, in some embodiments, the microfluidic channels of the microfluidic device 100 are fabricated from an elastic polymer that can optionally be coated on its interior surface to provide solvent resistance. The entire microfluidic channel or only a portion that is used as a valve system can be fabricated from an elastic polymer. In this design, the valve system can be electrically actuated. Such electric or pneumatic actuation of microfluidic devices is well known to one skilled in the art. By actuating an area of the elastic polymer, e.g., electrically, the actuated portion can be made to collapse onto itself, thereby closing the microfluidic channel. Such actuation prevents the fluid within the microfluidic channel from flowing. By continuously actuating one particular section of the microfluidic channel, one can close the microfluidic channel, thereby preventing flow of fluid within the microfluidic channel. Alternatively, microfluidic channel can be made to open when actuated.

When the microfluidic channels is made from an elastic polymer, it can include electrically activated system whereby a peristaltic-like wave movement can be created causing the fluid within the microfluidic channel to move in the direction of peristaltic-like wave. Thus, in this design, there is no pump unit 244. Rather the solution chamber valve system and the pump is an electric activator that can cause the fluid within the microfluidic channel to flow or stop flowing. For example, by continuously actuating one particular section of the microfluidic channel, one can close the microfluidic channel, thereby preventing flow of fluid within the microfluidic channel.

In one particular embodiment, the valve systems 236A, 236B, 240A, 240B, 252A, and 252B comprise a polymer or a material (e.g., wax) that is heat sensitive. In this manner, by heating such a material will cause the material to melt flow into (e.g., microfluidic channel closing valves) or away (e.g., microfluidic channel opening valves) from the microfluidic channel. Such valve systems are well known to one skilled in the art. See, for example, Hopwood et al., Anal. Chem., 2010, 82, 6991-6999, and Estes et al., Analyst, 2012, 137, 5510, which are incorporated herein by reference in their entirety.

In some embodiments, the microfluidic device 100 is fabricated or produced from a material comprising a polymer such as polycarbonate, polyacrylate, polymethacrylate, and other suitable polymers known to one skilled in the art. The microfluidic device 100 can also be fabricated from a material comprising a copolymer. Suitable copolymers useful for a particular application of microfluidic device 100 are also well known to one skilled in the art.

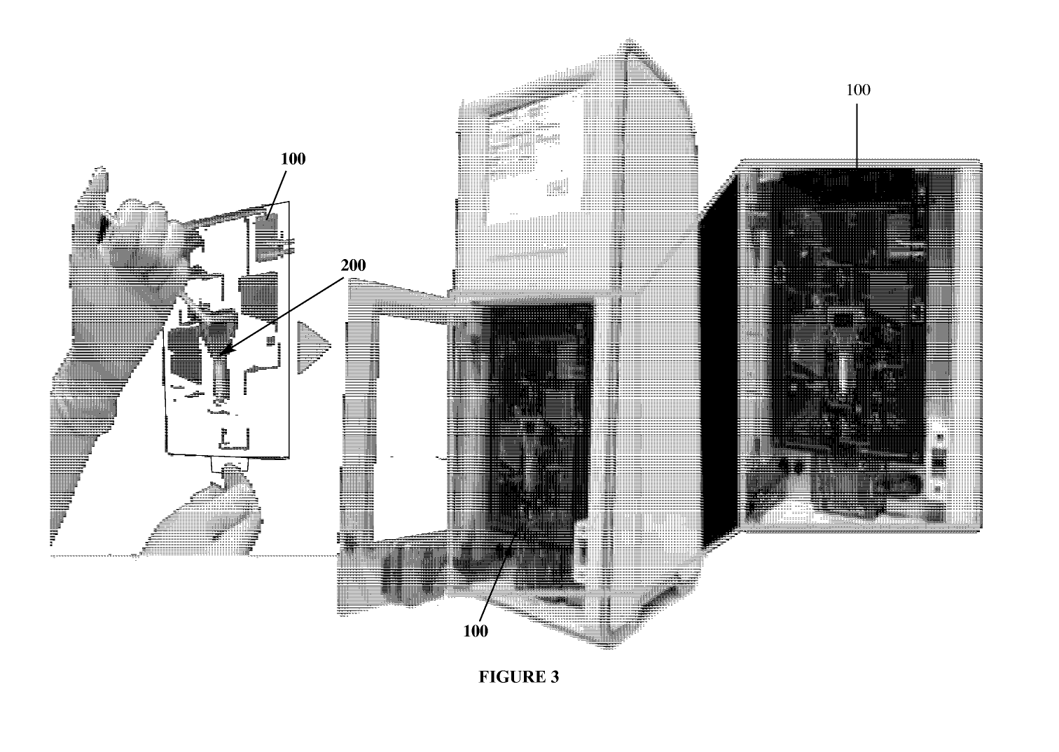

FIG. 3 shows use of a microfluidic device 100 in an instrument such as a miniaturized processing and controlling hardware system (i.e., “mini-MiDAS”). The mini-MiDAS instrument (the middle image) is 19″ (height)×10″ (width)×8″ (depth), with an inside operating area of 9″×8″ (the right image: cropped area). The left image shows loading of swab head into material processing chamber on the microfluidic device 100. Once the material is loaded onto the material processing chamber 200, the microfluidic device 100 is placed in the mini-MiDAS instrument (middle image). Extraction of a biological sample is then conducted automatically by mini-MiDAS instrument having an appropriate program algorithm.

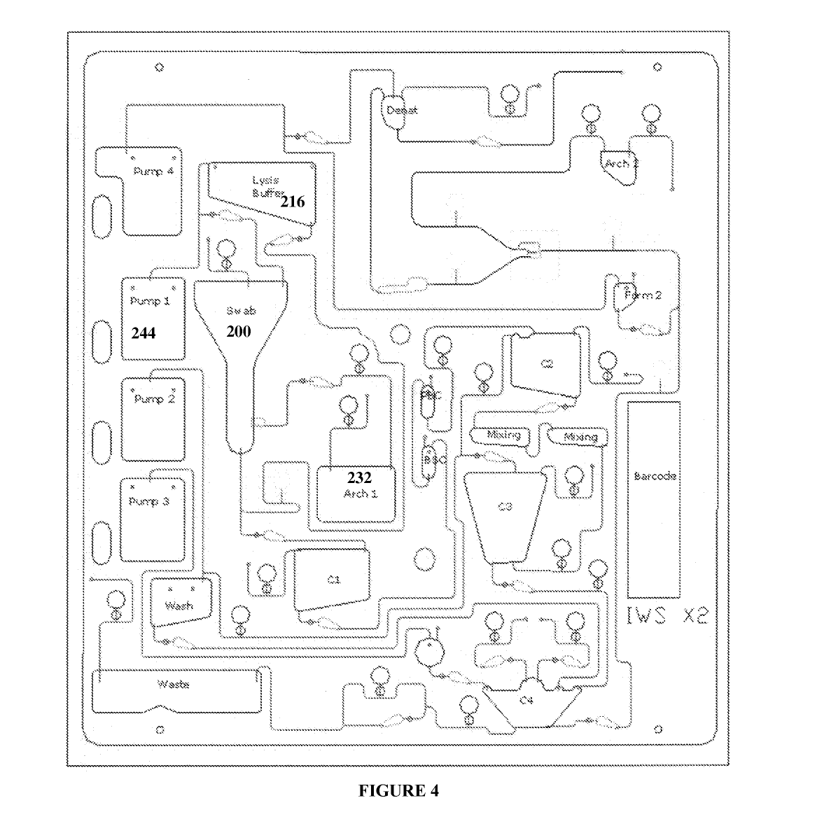

FIG. 4 shows a schematic drawing of one particular embodiment of the microfluidic device 100 of the invention that includes integrated downstream processing elements. The microfluidic device 100 illustrated in FIG. 4 is particularly suitable for swab sample processing. However, it should be appreciated any solid material can be processed using the microfluidic device 100 shown in FIG. 4. In this particular embodiment, the microfluidic device 100 includes swab processing module (which includes element 200) to obtain a biological sample from the swab, DNA lysis module (C1), DNA extraction module (C2 and C3), multiplex PCR module (C4) and transfer module delivering PCR product to the CE microchip for CE detection. The following describes a typical use of the microfluidic device 100 in FIG. 4. A buccal swab is placed into the “key-shaped” swab lysis chamber 200. One ml of lysis buffer is delivered into the lysis chamber 200. The lysate is then metered 150 mL to 200 mL in the lysate chamber, followed by DNA extraction, PCR and CE, as described in Hopwood et al., Anal. Chem., 2010, 82, 6991-699. The excess of lysate can be archived in the lysate archive chamber 232, which can be collected to perform a bench top control analysis with the same swab sample.

In some embodiments, the microfluidic device 100 can be integrated with other downstream processing units. In one particular embodiment, the microfluidic device 100 is a plastic cartridge module capable of integrating processing of forensic swab samples for a biological sample comprising DNA, lysing the extracted biological sample to obtain DNA, and delivering the lysate to the chamber which can be integrated with a downstream microfluidic device for DNA purification, amplification, and detection. In some embodiments, as shown in FIG. 4, one of more of these processes can be integrated within the microfluidic device 100. Microfluidic device of the invention offers the advantage of simple design, easy fabrication, cost-effectiveness, disposability and reduction of one more manual step for potential contamination. The microfluidic device of the invention can process a variety of common forensic sample types, including buccal, blood, and saliva swabs, and its suitability to different lysis buffer conditions from several commonly accepted chemistries was demonstrated as well.

By automating the extraction of DNA samples directly from solid materials, the microfluidic device and method of the invention eliminate the use of trained personal in sample preparation. Chemistries have been developed to improve the amount of DNA that is recovered from swabs. In some embodiment of the invention, the physical make-up of the swab has been modified to improve the amount of DNA released.

While a systems for purification, amplification, and detection of liquid samples already extracted from a solid material is currently available, no device and method are available that utilizes fully automated extraction process to obtain a biological sample from a material, in particular a solid material such as a swab. Thus, the device and method of the invention provide a simple sample-in answer-out functionality.

In some embodiments, the material processing chamber 200 for biological sample extraction from a solid material carries out the functions of heating and agitation required for improved biological sample extraction. The ability to carry out on-cartridge biological sample extraction from a solid material presents a significant step forward in the development of a fully automated sample-in answer-out system.

The following general process illustrates a typical procedure for using a microfluidic device 100 shown in FIG. 1B to extract a biological sample from a swab containing the biological sample. In one particular embodiment, the material processing chamber (C2 or 200) is integrated within a polycarbonate microfluidic cartridge. One particular embodiment of details on the specifics of cartridge assembly, pump and valve actuation, and the principle of passive valve actuation can be found in Hopwood et al., Anal. Chem., 2010, 82, 6991-6999. The following legends are generally applicable to FIG. 1B: P1=electrochemical pump; C1=buffer storage chamber for 1 mL volume; C2=swab lysis/elution chamber, C3=metered volume of 150 μl for downstream sample processing; Arch=storage chamber for 850 μl of excess crude DNA-containing lysate; 224=metering channel for 150 μl of lysate sample; OV1, OV2, OV3=opening valves; and CV1, CV2, CV3=closing valves.

The first step in traditional benchtop protocol for forensic buccal swab DNA extraction involves placing the swab in an Eppendorf tube filled with 1 mL of lysis buffer containing proteinase K and incubating at either room temperature or a temperature recommended by manufacture for 15 minutes. It was discovered by the present inventors that heating a swab (e.g., to 60° C.) improved the recovery of DNA. Thus, following 60° C. incubation the tube is vortexed to increase the quantity of DNA-containing lysate that is released from the swab. Thus, the following process for using the microfluidic device for sample extraction attempts to simulate these conditions as closely as possible.

The swab is inserted through the material inlet port 204 (i.e., C2) and the swab head is released into the cartridge. The inlet port 204 is then sealed using sterile PCR tape. The automated elution protocol is carried out by using an instrument. For example, in one particular elution protocol, without user interference, is provided below:
Activate OV1 (236A), that is activate a heating element to heat OV1 to cause the wax contained within 236A to melt, thereby opening the microfluidic channel 220 to allow flow of solution from 216 to 200;
Activate P1 (244). This allows transfer of an elution buffer from 216 to 200;
Deactivate OV1 (This simply turns the heating element off);
C2 is heated to 60° C.
Wait 15 minutes; It should be noted that P1 (244) continues to supply air to the bottom of C2 (i.e., 208), creating bubble mixing that gently agitates the swab/buffer mixture during the 15 minute incubation step, simulating the benchtop vortexing step and resulting in improved DNA recovery.
Return C2 to room temperature.
Activate CV1 (i.e., 236B). This closes the microfluidic channel 220.
Deactivate P1
Deactivate CV1.
Activate OV2. This opens microfluidic channel 256.
Activate P1
Deactivate OV2
Deactivate P1
Activate CV2. This closes the vent 248.
Deactivate CV2
Activate OV3. This allows transfer of the sample solution to the metering element 224 and the archiving chamber 232.
Activate P1. This causes flow of the sample solution from C2 to the metering element 224. The passive valve architecture of the metering element 224 ensures 150 μl of sample is captured and the excess 850 μl is delivered to the archive chamber 232.
Deactivate OV3
Deactivate P1
Activate CV3. This closes the preferential pathway from the material processing chamber C2 (i.e., 200) to the archive chamber 232.
Deactivate CV3
Activate P1.

With CV3 closed, the 150 μl in the metering element 224 is delivered to C3 (i.e., 264), a dead end chamber that represents the entry point to the further sample processing. It should be appreciated that other variations of the material processing procedures can also be used depending on the material being processes and/or the biological sample to be obtained.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### Examples

### Swab Lysis Cartridge Designs

The swab lysis cartridge was designed using the same fabrication format as the present inventors' existing Sample Prep-Polymerase Chain Reaction-Capillary Electrophoresis (SP-PCR-CE) system. Hopwood et al., Anal. Chem., 2010, 82, 6991-6999. It worked as a standalone functional module (FIG. 1A.) during its characterization process, lysing the swab material directly on the cartridge, and eliminating the off-chip lysis step. Design of this standalone module was also modified by adding a serpentine shaped metering element (FIG. 1B.) allowing a desired volume of lysate to meter into the lysate chamber (e.g., 150 μL lysate), to attain a similar lysate volume to the current input-lysate volume in the present inventors' existing SP-PCR-CE cartridge and achieved a total analysis system.

The swab head was placed into the central chamber via the top window 204 on chamber 2 (C2, i.e., material processing chamber 200) and then 1 ml of lysis buffer from 216 (i.e., C1) was introduced into the material processing chamber 200. The resulting mixture was agitated, and incubated for a period of time and under a certain temperature as the manufacturer's protocol recommended. During the incubation period, continuous bubbling was introduced from the bottom of the C2 chamber (i.e., 200) through 208 to assist the lysis process. The sample lysate was then transferred to chamber 3 (C3, i.e., the metering element 264) and the lysate archive chamber 232 which would be the raw sample lysate chamber in an integrated cartridge. The swab was maximally agitated by the steady introduction of micro-bubbles into the bottom of the chamber 200 during the incubation process.

The swab lysate was then retrieved from the metered sample chamber (C3, i.e., 264) to proceed with the on-benchtop 2^{nd} lysis step (see below—double lyses), DNA extraction and quantification for evaluating the lysis efficiency. The dye test was performed initially to ensure the functioning of the microfluidic flow-structure (data not shown).

The standalone microfluidic device of FIG. 1A was later redesigned by adding a metering capability to it. The serpentine structure was a metering element. Using varying fluidic resistances, the lysis buffer is first directed to a larger 1 mm×1 mm serpentine metering channel that leads toward further sample processing. Once that channel is filled with the designated volume (e.g., 150-200 μL buccal lysate) of lysate solution, a 0.25 mm×0.2 mm passive valve (240B) is reached that causes the lysis buffer to take the alternate path at the channel junction. This 1 mm wide, 0.5 mm deep channel directs all excess lysate to an archive chamber (232) where it can subsequently be collected. Once all lysate solution has been removed from the C2 chamber (200) and delivered to the archive chamber 232, the pathway to the archive chamber 232 is sealed with a valve 240B. The pump 244 is continuously engaged and the buildup of pressure is enough to breach the passive valve and direct the metered lysate solution on to the C3 chamber (264). This C3 chamber (264) serves to represent the lysate input chamber in the integrated SP-PCR-CE cartridge and can then be integrated into the rest of the sample workflow, performing sample preparation (SP), PCR amplification and release to the CE microchip. To demonstrate the compatibility of the swab lysis cartridge with further on-cartridge downstream processes, on-cartridge buccal swab lysis was evaluated in a preliminary study, followed by manually transferring 200 μL of buccal lysate from C3 chamber (264) of microfluidic device 100 into the crude lysate chamber in a sample prep cartridge. Using a MiDAS instrument, the downstream functionalities of SP and PCR were performed automatically. The PCR products were recovered post-amplification and analyzed on 3130 Genetic Analyzer for a standardized measurement and comparison.

Cartridge Fabrication and Instrumentation:

Similar to the other modules, the swab lysis element consists of computer numerical control (i.e., CNC)-machined channels, chambers, and valves that completely isolate the sample from the outside environment to significantly reduce the risk of cross-contamination. The use of small volumes and optimized procedures allows faster completion without sacrificing the sensitivity of the measurement. All the necessary reagents were pre-loaded onto the cartridge.

The fixture used for the test is a custom-built Sample Preparation/PCR unit (see FIG. 3). The Sample Prep/PCR unit includes internal power supplies, temperature controllers, a Sample Prep Board, and an onboard computer with monitor. The specially-designed circuit board called the Sample Prep Board has resistive heaters located where the microfluidic device's paraffin-based open and close valves (e.g., 236A, 236B, 240A, 240B, 252A, and 252B) are positioned and pogo-pin electrodes to connect to the electrochemical pump wires on the microfluidic device 100 (e.g., FIG. 1B). The board has a microcontroller to communicate with the onboard computer and execute control functions for the heaters and pumps. A temperature controller is also connected to the onboard computer and is used to set the required temperature for the swab lysis chamber (C2, i.e., material processing chamber 200). Onboard software was used to run the Sample Prep Board using a predesigned set of sequence instructions to automatically perform the test sequence for the sample preparation process.

Swab Sample Preparation:

Three common forensic sample types were used for preparation for swab samples: buccal, saliva and blood swabs. All the swab sample preparation was performed in a biological hood. The swabs used were Omni Swabs (Whatman, Maidstone, UK) with ejectable swab head. In an effort to limit variations in swab sample preparation, all swabs were prepared from an aliquot of sample directly. No dilutions were attempted.

Buccal Swabs—

Buccal samples were collected from one consenting donor (ID #AD) using an Omni Swab and following the Institutional Review Board (IRB) approved protocol. The swabs were obtained by rubbing inside the mouth cheek with the same numbers of strokes and motion of rubbing per buccal swab sample each time. The swab head was ejected into a sterile 1.5 mL polypropylene tube and stored in a −20° C. freezer till use.

Saliva Swabs—

Saliva was collected from one single donor (ID #AD) following the IRB approved collection protocol. In an attempt to limit sample to sample variation and ensure the reproducibility, several collections from the single donor were combined in a 50 mL polypropylene conical tube, mixed well, then an aliquot of 50 μL saliva mixture was deposited onto the sterile Omni swab with ejectable head. The swab was allowed to air dry at room temperature for 30 min before ejecting the swab head into a sterile 1.5 mL polypropylene tube. The swab sample tubes were stored in a −20° C. freezer until use.

Blood Swabs—

Normal human whole blood product was obtained from BioChemed Services (Winchester, Va.). In order to limit swab to swab variation, aliquots of blood were used for blood swab preparation, instead of from blood stain. 10 μL of the whole blood was deposited on each Omni swab head, allowing it to air dry for 30 min in hood. The blood swab head was then ejected into a sterile 1.5 mL tube and stored in a −20° C. freezer till use.

Swab Lysis and Protocols:

For on-cartridge swab lysis, the swab head was transferred from the storage tube into the key-shaped swab chamber 200 (FIG. 1B) with sterile forceps. The cells on the swab samples were directly lysed by flowing in 1 ml of lysis buffer from the C1 chamber (i.e., solution chamber 216) into the C2 chamber (i.e., material processing chamber 200) where the swab head was located. Three lysis buffer systems from three different DNA extraction kits were used to evaluate the swab lysis cartridge performance, i.e., ChargeSwitch gDNA Normalized Buccal Cell kit and ChargeSwitch Forensic DNA Purification Kit (Invitrogen, Carlsbad, Calif.), DNA IQ System (Promega, Madison, Wis.), and PrepFiler Forensic DNA Extraction Kit (Applied Biosystems, Foster City, Calif.). All of these kits were magnetic bead-based chemistries, for the ease of on-cartridge downstream sample prep integration. For each swab type and each lysis buffer system used, 5 lysis cartridges and 3 bench top controls were processed. A total of 45 swab lysis cartridges were evaluated.

For Invitrogen ChargeSwitch gDNA Normalized Buccal Cell Kit (CS) (Life Technologies, Carlsbad, Calif., USA), 1 mL lysis buffer plus 10 μL Proteinase K solution (provided in the kit) were used for lysis of the buccal swabs by incubating at 60° C. for 15 min with bubbling. For blood and saliva swabs, ChargeSwitch Forensic DNA Purification kit (CS) was used and the lysis conditions were 55° C. for 30 min and 60° C. for 15 min, respectively.

For DNA IQ System (DNA IQ) (Promega Corporation, Madison, Wis., USA), all swab samples were lysed in 1 mL of DNA IQ Lysis Buffer and 10 μL of 1M Dithiothreitol (DTT) and incubated at 70° C. for 20 min.

For PrepFiler Forensic DNA Extraction Kit (PF) (Life Technologies, Warrington, UK), all swab samples were lysed in 1 mL of PrepFiler Lysis Buffer and 10 μL of DTT solution, incubating at 80° C. for 15 min.

Controls were run in parallel on bench top in-tube swab lysis, with the same lysis conditions performed for on-cartridge lysis with each chemistry kit and for each swab sample type.

Double Swab Lysis:

In order to limit swab-to-swab variation, each swab was subjected to two rounds of lysis: the first one on-chip and the second one in a tube. After the 1st round of on-cartridge swab lysis, the sample lysate was collected from the chamber C3 (264) and the swab head was retrieved from the cartridge C2 chamber (200) with sterile tweezers, transferred into a new 1.5 ml tube where another 1 ml of fresh lysis buffer was added and the 1st lysis process was repeated in tube. The sample lysate from the 1st and the 2nd lyses were collected into new tubes and DNA was extracted from the lysate samples respectively. The combination of quant from both lysis steps equals the total DNA concentration obtained from one swab. Variation in the total DNA concentration obtained from swabs indicates the variation in biological material on the swab among swab samples, therefore ensuring the difference in lysis efficiency is not caused by the difference in biological materials on the swab. However, only the lysis efficiency from the 1st round of swab lysis was calculated and compared, while the result from the 2nd round of swab lysis was mainly used for normalization.

Genomic DNA Extraction and Quantification:

In order to exclusively evaluate on-cartridge swab lysis efficiency, all of the sample lysate collected (both 1st and 2nd lyses) went through a manual DNA extraction process with the same chemistry kit as used in the swab lysis steps. Three forensic DNA extraction chemistries were evaluated, ChargeSwitch gDNA Normalized Buccal Cell kit (for buccal swan only) and Forensic gDNA Purification kit, DNA IQ System, and PrepFiler Forensic DNA Extraction kit. Following swab lysis, the DNA in the sample lysate collected from both (1st and 2nd) lysis steps, was extracted manually and separately, quantified, and compared to controls. All DNA extractions were performed on benchtop, according to manufacturer's protocols provided in the kits. For ease of comparison, all extracted DNA samples were eluted in 150 μL of elution buffer, regardless of different extraction chemistries used.

During evaluation and optimization of swab lysis cartridge performance, DNA quantification of the DNA extracted from the sample lysates was performed by real-time PCR analysis, using the Quantifiler Human DNA Quantification Kit (Applied Biosystems, Warrington, UK) in accordance with the manufacturer's instruction and on Stratagene's Mx3005P instrument.

Calculation of the on-Cartridge Swab Lysis Efficiency:

In practice, only one lysis step is performed for each swab sample, therefore only the 1 st round of swab lysis efficiency, which was on-cartridge lysis, was calculated and compared with the 1st round of benchtop control swab lysis. To reduce swab to swab variation, every swab went through two rounds of lysis, with only the 1st round of lysis performed on-cartridge. The 2nd round of lysis was completed by retrieving the swab from the cartridge and placing in a tube with fresh lysis buffer. DNA extraction was performed manually on the two sample lysates collected. The total DNA Quant was the sum of DNA concentrations from 1st lysate and 2nd lysate. The lysis efficiency was calculated using the quant data. Each fraction (the 1st or the 2nd lysate) of the lysis quant was normalized to the total quant (i.e. the 1st+the 2nd) obtained for that swab. The more similar total quant results were from swab to swab, the less variation was obtained among swab samples.

A formula for lysis efficiency (%) calculation:

$$Lysis Efficiency = \frac{The {1}^{st} lysis \text{/} Extraction Quant}{The total Lysis \text{/} Extraction ( {1}^{st} + {2}^{nd} ) Quant} \times 100 \%$$

1^{st} Lysis efficiency=[Quant of 1^{st} Lysate-extraction/total DNA Quant (=1^{st}+2^{nd} DNA Quants)]×100%

2nd Lysis efficiency=[Quant of 2^{nd} Lysate-extraction/total DNA Quant (=1^{st}+2^{nd} DNA Quants)]×100%

STR Genotyping—3130 Analysis:

To ensure the quality of the on-cartridge lysed samples, the DNA samples extracted from the swab lysates were amplified with PowerPlex ESI17 amplification multimix (Promega, Madison, Wis.) and a rapid 27 cycle amplification reduced protocol modified from the manufacturer's parameters for application on the integrated sample-to-answer system. Reduced, 10 μL reaction volumes were used, with 1 μL of eluted DNA for amplification, except for blood swab samples which used 3 μL, 5 μL, and 6 μL of eluted DNA in the PCR reactions from blood swab samples extracted with ChargeSwitch (CS), DNA IQ, and PrepFiler (PF) chemistries, respectively. The volumes used were based on the quantification results, in order to get the amplified peak heights in an optimal range for analysis. The PCR products were separated on Applied Biosystems 3130xl instrument in accordance with the PowerPlex ESI 17 kit instructions, for STR profile analysis. Electrophoresis data were analyzed using GeneMapper ID v3.2.1 software (Applied Biosystems).

Integration of Swab Lysis Module with Our Existing Sample Prep-PCR-CE System:

Integration of this swab lysis module with downstream DNA extraction and PCR was demonstrated by its compatibility with an existing rapid DNA analysis system—MiDAS. Briefly, the buccal swab was lysed on the swab lysis cartridge, on the mini-MiDAS instrument (FIG. 3), following the Manufacturer's protocol. 200 μL of the lysate metered into the designated sample lysate chamber 264 (FIG. 1B) was transferred manually into the raw lysate chamber on an integrated sample prep cartridge (Figure not shown) where DNA extraction (with ChargeSwitch gDNA Normalized Buccal Cell kit) and multiplex PCR (with PowerPlex ESI17 kit were performed automatically. Amplified product was retrieved from its chamber and the capillary electrophoresis (CE) separation of the post-PCR product was performed on 3130xl Genetic Analyzer (Applied Biosystems), in order to standardize the results analysis.

### Results and Discussion

Feasibility Study and Cartridge Modification:

The design of swab lysis cartridge 100 was simple in fabrication, can be integrated with other downstream on-cartridge functionalities in microfluidic workflow, and functionally compatible with benchtop control in lysis efficiency. Upon completion of cartridge fabrication, colored dye tests were performed (data not shown) to ensure all the functional elements (chambers, channels, valves, pump, bonding, etc.) were working properly. To facilitate the adequate recovery of biological material from the swab matrix, an active bubble-mixing was applied during the swab lysis incubation, instead of relying on passive diffusive mixing. Several iterations of cartridge modifications were made to ensure there was adequate volume of lysis buffer covering the swab head, the contact between printed circuit board (PCB) and cartridge was tight, the amount of bubbling to agitate the lysis buffer was sufficient, and the valves closed/opened properly.

In the feasibility study, buccal swab samples were used. The DNA samples extracted from either the on-cartridge lysis tests or benchtop lysis controls, were quantitated using Stratagene Mx300SP. Results from 5 independent lysis experiments with 2 controls in-tube lysis and 3 on-cartridge lysis were analyzed. Since the 2nd round of lysis for both on benchtop and on-cartridge were performed in-tube on-benchtop, the only variable was the 1st round of swab lysis, i.e., either on-cartridge or on-benchtop. Although the quant results showed the concentration of lysed material in the 1st and/or 2nd lysate solutions for either on-cartridge (2.3 ng/μL and 0.6 ng/μL) or on-benchtop (1.5 ng/μL and 1.25 ng/μL), were different, the total DNA concentration (the sum of 1st and 2nd lyses per swab) was comparable between on-cartridge and on-benchtop lyses, indicating the difference observed in the 1st swab lysis efficiency was independent of varying swabs. The results show the on-cartridge lysis was, if not better, as efficient as the traditional off-chip lysis.

The quality of DNA samples extracted from on-cartridge lysed buccal swab lysate was confirmed by STR analysis on 3130xl Genetic Analyzer. 3 μL of extracted DNA was used for PowerPlex ESI17 amplification. Full profiles were obtained with balanced allelic peak height and tail-off ratios (data not shown).

During the preliminary test, buccal swab samples were used before conducting a thorough evaluation for lysis efficiency with different swab samples—blood swabs, saliva swabs and buccal swabs.

Evaluation of Swab Lysis Cartridge Efficiency with Different Swab Sample Types and Lysis Reagents:

Following the feasibility study, the performance efficiency of the swab lysis cartridge was evaluated using different forensic sample swabs (buccal swabs, blood swabs, and saliva swabs), and different sample lysis/DNA extraction chemistries (CS, DNA IQ, and PF) were used to extract DNA from the lysed materials, to examine the compatibility of the swab lysis cartridge with different lysis buffer systems. The three chemistry systems selected were all magnetic bead-based technology to isolate genomic DNA without the need for centrifugation or vacuum manifolds. Typically, about 700 μL of lysate solution was retrieved per one ml swab lysis buffer, regardless of chemistry used, which was subsequently subjected to DNA extraction directly.

Swab Lysis Efficiency with CS Lysis Buffer and DNA Extraction Kit:

ChargeSwitch (CS) beads provide a switchable surface charge dependent on the pH of the surrounding buffer to facilitate nucleic acid purification. Two kinds of CS forensic DNA extraction kits were used. The CS Normalized gDNA Buccal Cell kit was used exclusively for human buccal swabs, while blood swab and saliva swab samples was processed with CS Forensic DNA Purification kit. Comparing to other chemistries, CS reagents were easier in incorporating onto the cartridge platform due to 1) the CS bead size is relatively larger and doesn't require extremely high external magnetic force for the capture; 2) less foam formation during bubble-agitation in the mixing step, 3) reagents do not contain hazardous chemicals and are compatible with the cartridge material; and 4) less siphoning effect during fluid transfer in the microfluidic device.

Following the feasibility study, different types of swab samples were examined with CS lysis buffer. Both buccal and saliva samples were obtained from the same volunteer donor, except for the human blood sample (BioChemed, Winchester, Va.). Except the first round of swab lysis, the 2nd round of swab lysis and all remaining extraction steps following lysis were done manually on benchtop. Therefore, only the 1st swab lysis step was variable and all remaining steps were standardized with benchtop procedure. Comparing the 1st lysis efficiency, the on-cartridge swab lysis was very comparable to the benchtop in-tube swab lysis for the buccal and saliva swab samples and even better in the saliva swab lysis.

However, in the blood swabs lysis with CS chemistry, the on-cartridge swab lysis under-performed in comparison to its benchtop controls. Table 1 shows the quant results from the 1st swab lysis/DNA extraction samples, for all three swab sample types and lysed with three different lysis buffer systems. Due to the nature of buccal swabbing, the number of epithelial cells collected with each buccal swab varies. Therefore the same donor was volunteered for all the buccal swabs, trying to prepare the buccal swabs in a consistent manner. The DNA yields obtained from the 1st swab lysate (the 1st lysis elution) were 17.7±6.9 ng/μL (n=5) for on-cartridge lysed buccal samples; 3.5±0.6 ng/μL (n=5) from on-cartridge lysed saliva swab samples; and 1.2±0.5 ng/μL (n=5) from on-cartridge lysed blood swab samples, respectively. The results of DNA yield achieved from on-cartridge (i.e., microfluidic device 100) buccal and blood swab lyses were similar to the findings published, in evaluating an enzyme-based DNA preparation method for non-degraded buccal swab and blood samples and swab samples run on a commercial instrument, although the DNA concentration from the on-cartridge lysed blood swab was about 60%-70% lower than that obtained from blood swab lysed in-tube (Table 1).

TABLE 1

| Extraction | Swab Lysis | Number of | Mean [DNA](ng/μL) ± STDEV | | |
|---|---|---|---|---|---|
| Chemistry | Methods | Replicates | Buccal Swab | Saliva Swab | Blood Swab |
| ChargeSwitch | Benchtop | 3 | 12.3 ± 1.9 | 5.9 ± 2.5 | 3 ± 0.9 |
| | On-Cartridge | 5 | 17.7 ± 6.9 | 3.5 ± 0.6 | 1.2 ± 0.5 |
| DNA IQ | Benchtop | 3 | 2.5 ± 0.4 | 4.8 ± 0.1 | 0.3 ± 0.1 |
| | On-Cartridge | 5 | 3.3 ± 1.1 | 6.3 ± 1.2 | 0.3 ± 0.1 |
| PrepFiler | Benchtop | 3 | 14.3 ± 2.4 | 8 ± 2.9 | 0.9 ± 0.4 |
| | On-Cartridge | 5 | 12.5 ± 9.8 | 12.9 ± 2.5 | 0.5 ± 0.1 |

Swab Lysis with DNA IQ System Lysis Buffer:

Using the DNA IQ System, the swabs were lysed with DNA IQ Lysis Buffer under the recommended conditions by the manufacturer. The quantitative comparison showed that the on-cartridge swab lysis (the 1^{st} swab lysis) efficiency was similar or equal to their benchtop controls for buccal and saliva swab samples, except for blood swab sample which showed a lysis efficiency about 15% lower than its in-tube control. The DNA concentrations were 3.3±1.1 ng/μL and 6.3±1.2 ng/μL, for on-cartridge lysed buccal swab samples (n=5) and saliva swab samples (n=5), respectively. Similar to the finding with CS chemistry, the DNA yield obtained from blood swabs appeared low in quant from both on-cartridge lysis and benchtop in-tube lysed samples (Table 1), independent of where the swab lysis was performed. The average DNA yield obtained from blood swabs was 0.3±0.1 ng/μL (n=5). Since the extracted DNA was eluted in 150 μL elution buffer, the averaged total DNA yield from blood swabs was about 45 ng, comparable to the result obtained from the commercial instrument. For both buccal and saliva swab samples, the performance of on-cartridge lysis was very comparable to their benchtop controls, in both DNA extraction and lysis efficiency.

Swab Lysis with PrepFiler (PF) Lysis Buffer:

It appeared that the swab lysis efficiency (mainly the 1st swab lysis) using PrepFiler (PF) lysis buffer and extraction kit was similar to the other two chemistry kits (CS and DNA IQ) across all three swab sample types. For the blood swab samples, PF chemistry performed well on benchtop in-tube lysed/extracted samples, with a lysis efficiency of 88.3±4.7. However, the DNA yield from on-cartridge lysed blood swab lysate appeared low (0.5±0.1 ng/μL), comparing to buccal and saliva swab samples (12.5±9.8 ng/μL and 12.9±2.5 ng/μL, respectively) (Table 1). The total DNA obtained from the on-cartridge lysed blood swabs in 150 μL elution buffer ranged from 60 ng to 90 ng, with an average DNA concentration of 75±15 ng. The significantly lower DNA recovery efficiency from the blood swab with PF chemistry might be explainable by the findings reported for automated PF protocol processed blood swab sample on the commercial instrument.

During the characterization of swab lysis cartridge (microfluidic device 100), a double swab lysis approach was applied. By performing the 2nd lysis step on each swab, data can be normalized by maximizing the total DNA yield per swab sample and therefore calculate the on-cartridge (the 1st lysis) lysis efficiency more accurately. From the total DNA yield, the swab-to-swab variations was evaluated. The DNA yield from the 2nd lysis indicated incomplete elution of biological sample from the swab heads after the 1st lysate was removed.

In summary, with the findings obtained from three forensic swab sample types using three magnetic bead-based DNA extraction chemistry lysis buffers, 1) comparing the on-cartridge swab lysis efficiency, buccal and saliva swabs had better performance than blood swab samples and CS chemistry on-cartridge lysed samples showed better and more consistent results in swab lysis efficiency across all three swab sample types among the three DNA extraction kits; 2) comparing the quant results among three swab sample types, the yield from human blood swab (10 μL per swab) gave the lowest DNA concentration across all three chemistry lysed/extracted swab samples, regardless of being on-cartridge swab lysis or their benchtop controls. It was a bit surprising that the blood swab (with 10 μL whole blood deposited on) had a relatively low yield as compared to the results others reported, especially in the case of DNA IQ extracted samples. It was confirmed that the blood product was not diluted in any way. Taking the total elution volume into consideration, results of DNA recovery from lysed swab samples were similar to the findings reported by others, for buccal swab, blood swab and saliva swab samples with different DNA extraction chemistries; 3) In order to compare the on-cartridge lysis efficiency with its benchtop in-tube lysis control, less variation in the biological material on each swab was desired for the valid comparison. Table 1 summarized the mean DNA yield collected from the 1st lysate of each swab sample, across three lysis buffer and extraction systems. Except for the CS and PF lysed/extracted blood swab samples where the DNA yield from on-cartridge lysed samples were lower than their benchtop controls, the DNA extracted from on-cartridge lysed swab samples was very comparable to that lysed in-tube on benchtop, regardless of swab sample types and lysis buffer chemistry used. It was observed that a higher standard deviation was associated with on-cartridge processed samples (Table 1), although in those cases the on-cartridge lysed swab samples did tend to have a higher DNA yield (i.e. CS and PF chemistries lysed buccal swabs; PF chemistry lysed saliva swab) and a high variation in DNA concentration recovered from buccal swabs was expected.

Quality of Swab DNA Samples from on-Cartridge Lysis:

The quality of DNA samples from on-cartridge lysed swab lysate was examined with STR genotyping analysis. All extracted DNA samples from the swab lysates, both from either benchtop or on-cartridge lysed swab sample lysates, were subjected to PCR using 3 μL PowerPlex ESI 17 regular liquid reagents (kit lot #318871) with 2:1 Master Mix to Primers ratio and profiled on the 3130xl Genetic Analyzer. The volumes of DNA template used for amplification were 1 μL, except for the blood swab samples which were amplified with a different volume for each chemistry. Various volumes of the eluted blood swab DNA samples were used in PCR reactions based on the quantification data, in order to keep the amplified peak heights for both benchtop and on-cartridge lysed samples in the range of 1000-4000 (rfu), without going too much out of the optimal detection range. Mean peak height value is calculated by dividing total profile peak height in relative fluorescent units (rfu) by a possible 34 peaks for PowerPlex ESI17 amplification.

Mean profile peak height values for the amplified DNA samples obtained from three different swab sample types (i.e., Buccal, 50 μL Saliva, and 10 μL Blood) lysed and DNA extracted using three different chemistries, CS, DNA IQ and PF, with on-cartridge and on-benchtop in-tube lysis samples compared, are shown in Table 2. Due to the lower DNA concentration(s) achieved from the blood swabs, blood samples were amplified using a higher volume of template (3 μL, 5 μL, and 6 μL for CS, DNA IQ, and PF lysed/extracted DNA samples, respectively, instead of 1 μL template used for buccal and saliva DNA samples), given that the 1 μL volume used for saliva and buccal extractions produced peaks for the blood samples that fell too far below the 1000-4000 (rfu) range for accurate analysis of profile quality. The mean was calculated by dividing total profile peak height intensity, in relative fluorescence units, by a possible 34 peaks for Power Plex ESI17 amplification. Replicates were averaged for each sample type and standard deviations between extraction replicates were shown as well. The mean peak heights for all three types of swab samples were very comparable between on-cartridge lysed samples and their benchtop controls for DNA IQ chemistry lysed/extracted samples (Table 2), with 2118±558, 2829±370, and 1072±266 (rfu) for on-cartridge lysed swab samples as compared to controls of 2743±636, 2843±9 and 1259±237 (rfu), for buccal, saliva and blood swabs respectively. In the CS chemistry lysed/extracted samples, the mean peak heights from the on-cartridge lysed buccal and saliva swab samples were similar to their in-tube lysed swab sample controls, except for the blood swab in which the low profile intensity (1015±161 rfu vs. 2409±102 rfu, for on-cartridge lysed sample and in-tube lysed sample, respectively) could be attributed to the lower template concentration for amplification (1.2±0.5 ng/μl), as compared to its benchtop control (3±0.9 ng/μl) (Table 1). Similarly, a lower mean peak height found in PF chemistry for on-cartridge lysed/extracted blood swab samples (827±121 rfu) was correlated with relatively lower DNA template concentration (0.5±0.1 ng/μl) than the corresponding benchtop lysis controls (0.9±0.4 ng/μl). Electropherograms (EPGs) were obtained from the PowerPlex ESI17 kit amplified PCR products. The DNA templates were extracted from the swab samples which were lysed on the swab lysis cartridge with different chemistry lysis buffers.

TABLE 2

| | | Mean Peak Height (rfu) ± STDEV | | | |
|---|---|---|---|---|---|
| Extraction | Swab Lysis | Number of | | | |
| Chemistry | Methods | Replicates | Buccal Swab | Saliva Swab | Blood Swab |
| Charge Switch | Benchtop | 3 | 2945 ± 178 | 1446 ± 33 | 2409 ± 102 |
| | On-Cartridge | 5 | 2640 ± 707 | 1561 ± 472 | 1015 ± 161 |
| DAN IQ | Benchtop | 3 | 2743 ± 636 | 2843 ± 9 | 1259 ± 237 |
| | On-Cartridge | 5 | 2118 ± 558 | 2829 ± 370 | 1072 ± 266 |
| PrepFiler | Benchtop | 3 | 3094 ± 436 | 1843 ± 92 | 1719 ± 505 |
| | On-Cartridge | 5 | 1656 ± 630 | 2129 ± 574 | 827 ± 121 |

Not only were the electropherogram (EPG) intensities of each sample STR profile analyzed, but the profile balance was checked by calculating the tail-off and intra-color ratios across the full profiles as well. The tail-off ratios averaged for three different swab sample types, i.e. buccal, saliva and blood swabs, respectively, and plotted by different lysis buffer systems, i.e. CS, DNA IQ and PF chemistries.

The quality of the on-cartridge lysed samples was assessed by the quality of the DNA samples extracted and amplified from the lysate solutions of on-cartridge lysed swab samples. The Tail-off Ratios were calculated by dividing the peak height sum of the longest fragments by the peak height sum of the shortest base number fragments, in each dye color, e.g., the total peak height of the Fluorescein dye-labeled D22S1045 peaks divided by the total peak height of the Fluorescein-labeled AMELO peaks. Ratios were averaged for replicates of each sample type and the standard deviations were graphed as well. By performing tail-off analysis of the multiplex STR PCR products, one can evaluate if the sample has: 1) DNA degradation; 2) too high or too low the DNA template concentration; 3) PCR inhibitor presence. The ideal ratio is “1.0” with values greater than or less than “1.0” showing a greater imbalance, though typically imbalance is characterized by value less than “1” (ski-slop effect) due to sample degradation or high DNA template concentration. The tail-off ratios for CS, DNA IQ and PF chemistries used in swab lysis and extraction, with three different swab sample types (i.e., buccal swab, swab with 50 μL Saliva, and swab with 10 μL Blood) were compared between on-cartridge and benchtop in-tube lysis.

Regardless of what chemistry kit was used, the profile balance from the on-cartridge lysed swab samples was comparable with, if not better than, their counterpart benchtop controls with ratios remaining above 0.5 across the board. There was a pattern of the saliva and buccal swab samples tending to have ratios a little below the optimal ratio of “1.0”, while most of the ratios for blood swab samples were slightly above this value. In addition, the Intracolor balance ratios were also calculated, i.e., within the same color dye(s), the sum of the lowest peaks divided by the sum of highest peaks, Independent of their fragment length.

The Intracolor balance ratio is another indicator for the quality of the DNA samples lysed/extracted. The intracolor balance ratios obtained from our on-cartridge lysed swab samples were comparable with that from in-tube lysed swab sample controls, suggesting that there is no significant degradation of template or amplification inhibition introduced by the on-cartridge lysis procedure. The CS lysed/extracted sample balance appeared to be more uniform than the other chemistry lysed samples, with higher overall ratios and with more consistent values between dye colors for each set, at least for Fluorescein, JOE and TMR-ET. The ratio values for CXR-ET dye are consistently lower than other dye colors, across all three lysis/extraction chemistries, which could possibly be improved by using the PowerPlex ESI17 Pro System released recently from Promega with newly designed primers for the SE33 locus.

In summary, swab lysis cartridge (microfluidic device 100) study showed that 1) for the on-cartridge swab lysis performance, DNA IQ chemistry lysed/extracted DNA samples demonstrated comparable lysis efficiency, quantification results and mean peak height values between on-cartridge lysed swab samples and on-benchtop in-tube lysed swab samples (Table 2), for all three different types of swabs—blood, saliva and buccal; 2) buccal swab samples demonstrated the highest and consistent pattern of efficiency across all three chemistries, while the saliva swab lysis/extraction samples demonstrated very comparable results between on-cartridge and benchtop efficiency, mean peak height and quantification results for all chemistries tested; 3) though the blood swab samples had a different pattern of tail-off ratio than saliva and buccal samples, all sample types exhibited relatively consistent ratios between benchtop and on-cartridge lysed samples across all chemistries used.

Integratable Swab Lysis Cartridge:

In alignment with the existing integrated SP-PCR-CE cartridge platform, the present inventors have designed, fabricated, and tested the swab lysis cartridge module (i.e., microfluidic device 100).

The swab lysis module is integration ready and compatible with the integrated sample prep-PCR-CE system (MiDAS Instrument). Due to the requirement for instrument modification, the initial integration evaluation was tested by a manual transfer of the on-cartridge lysed sample solution into the lysate chamber of MiDAS cartridge for extraction and amplification. The post-PCR product was analyzed on a 3130xl Genetic Analyzer, in order to standardize the results analysis and compare with benchtop controls. The benchtop controls were conducted with the same lysate as on-cartridge extraction, but with DNA extraction and PCR carried out manually and on a commercially available PCR instrument, respectively. The lysate used for each DNA extraction was 200 μL, rather than the whole 1 ml lysate used by most other groups, which would demonstrate higher sensitivity for our integrated system. The electrophoregram (EPG) was obtained from the on-cartridge buccal swab lysis, on-cartridge DNA extraction, using CS Normalized Buccal Cell kit, and on-cartridge amplified PCR products.

The on-cartridge lysed and extracted DNA samples were retrieved from the archive chamber with 113 μL and 118 μL, from the two cartridges respectively. The quantity of the purified DNA samples measured were 0.57 ng/μL and 0.53 ng/μL, or the total DNA concentrations extracted from 200 μL of buccal lysate were 64.4 ng and 62.5 ng, for buccal cartridges one and two, respectively. The quantification results from benchtop manual controls for the on-cartridge extraction was 2.3 ng/μL and 1.1 ng/μL, two to four times higher than the on-cartridge extracted samples, indicating some sample loss from the on-cartridge processed samples (by the fact that the cartridge has a larger surface area and inner surface smoothness affects the particles retention, etc.). With only 200 μL of buccal lysate, a full, relatively balanced STR profile was successfully demonstrated, indicating the swab lysis cartridge module is integratable not only in microfluidic workflow but also in its total analysis functionality.

In an attempt to achieve total integration from swab sample going-in to profile coming-out, the step of on-benchtop in-tube swab lysis and manually transferring the lysate to SP-PCR-CE cartridge needs to be eliminated. In alignment with current cartridge platform, the present inventors have designed, fabricated, and tested a swab lysis cartridge module, which can be integrated to MiDAS System. During the characterization of swab lysis cartridge, double swab lyses approach was applied. By performing the 2nd lysis step on each swab, data can be normalized by maximizing the total DNA yield per swab sample, and therefore calculate the on-cartridge (the 1st lysis) lysis efficiency more accurately. From the total DNA yield (i.e., the sum of quant from the 1st and 2nd lysed/extracted samples), the swab-to-swab variations were evaluated. The DNA yield from the 2nd lysis indicated incomplete elution of biological material from the swab heads.

The swab lysis cartridge was designed using the same fabrication process as the present inventors' existing sample prep (SP)-PCR-CE workflow, adding a front-end sample introduction module that processes the swab material directly and eliminates the off-chip lysis step. Comparing to automated commercial robotic instrument for forensic DNA extraction from cotton swab samples, results from using microfluidic device 100 were very comparable, in terms of DNA yield and quality of the integrated on-cartridge process from swab lysis to post-amplification product.

The microfluidic sample preparation device of the invention can be integrated to the cartridge-based microfluidic SP-PCR-CE STR genotyping systems starting with different forensic swab samples and compatible to different sample prep chemistries. The on-cartridge lysis efficiency was comparable with in-tube lysis controls, the DNA extracted from the on-cartridge lysed swab samples was shown to have good quality and quantity, and the design can be easily configured to accommodate most of commercial reagents and sample volume requirements.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A microfluidic sample preparation device (100) comprising:
a material processing chamber (200) adapted for extracting a biological sample using a solvent, said sample processing chamber (200) comprising:
a material inlet port (204);
a solution inlet port (208); and
a processed sample solution outlet port (212);
a solution chamber (216) adapted for holding a fluid comprising:
a solution delivery microfluidic channel (220) operatively connected to said solution inlet port (208), wherein said solution delivery microfluidic channel (220) is adapted for delivering a sample processing solution from said solution chamber (216) to said sample processing chamber (200);
a sample solution metering element (224) adapted for metering a desired volume of the sample solution to a sample analyzer, wherein said sample solution metering element (224) comprises:
a sample solution delivery microfluidic channel (228) operatively connected to said sample solution outlet port (212), wherein said sample solution delivery microfluidic channel (228) is adapted to provide a desired volume of the sample solution to a sample analyzer,
an excess sample solution storage chamber (232) operatively connected to said sample solution metering element (224), wherein said excess sample solution storage chamber (232) is adapted to store any excess portion of the sample solution; and
a pump element (244).

2. The microfluidic sample preparation device of claim 1, wherein said solution chamber (216) further comprises a solution chamber valve system for opening (236A) and closing (236B) said solution delivery microfluidic channel (220).

3. The microfluidic sample preparation device of claim 1, wherein said sample solution metering element (224) further comprises a sample solution metering valve system (240A and 240B) operatively connected to said sample processing chamber (200).

4. The microfluidic sample preparation device of claim 1 further comprising an electrochemical pump (244) that is operatively connected to said solution chamber (216) and is adapted to transfer a fluid from said solution chamber (216) to said sample processing chamber (200).

5. The microfluidic sample preparation device of claim 1 further comprising an electrochemical pump (244) that is operatively connected to said sample processing chamber (200) and is adapted to transfer the sample solution from said sample processing chamber (200) to said sample solution metering element (224).

6. The microfluidic sample preparation device of claim 1, wherein said sample processing chamber (200) further comprises a vent (248).

7. The microfluidic sample preparation device of claim 1, wherein said device is a cartridge.

8. The microfluidic sample preparation device of claim 1, wherein said sample processing chamber (200) is adapted to provide an active mixing of the sample and the sample processing solution.

9. A method for processing a solid material comprising a biological sample using a device of claim 1 to obtain the biological sample therefrom, said method comprising:
(i) placing a solid material comprising a biological sample in said sample processing chamber (200) via said sample inlet port (204);
(ii) activating said solution delivery microfluidic channel (220) thereby transferring a sample processing solution from said solution chamber (216) to said sample processing chamber (200);
(iii) processing the sample in said sample processing chamber (200) under conditions sufficient to produce a desired biological sample solution from the solid material; and
(iv) activating said sample solution delivery microfluidic channel (228) thereby transferring the sample solution to said sample solution metering element (224).

10. The method of claim 9, wherein said steps (ii)-(iv) are automated.

11. The method of claim 9 further comprising the step of transferring an excess sample solution to said excess sample solution storage chamber (232).

12. The method of claim 9, wherein said device further comprises a pump (244) that is operatively connected to said solution chamber (216), and wherein said step (ii) of activating said solution delivery microfluidic channel (220) comprises activating said pump (244) thereby transferring the fluid from said solution chamber (216) to said material processing chamber (200) through said solution delivery microfluidic channel (220).

13. The method of claim 9, wherein said device further comprises a pump (244) that is operatively connected to said material processing chamber (200), and wherein said step (iv) of activating said sample solution delivery microfluidic channel (228) comprises activating said pump (244) thereby transferring the sample solution from said material processing chamber (200) to said sample solution metering element (224) through said sample solution delivery microfluidic channel (228).

14. The method of claim 9, wherein said device is a cartridge.

15. The method of claim 9 further comprising the steps of transferring the sample solution in said sample solution metering element (224) to an analytical device to analyze said sample solution.
